(19) United States
(12) Patent Application Publication
(10) Pub. No.: US 2025/0195600 A1
(43) Pub. Date: Jun. 19, 2025

(54) COMPOSITION FOR PREVENTION OR TREATMENT OF NEUROINFLAMMATION, CONTAINING DAPHNE GENKWA FLOWER BUD EXTRACT AS ACTIVE INGREDIENT

(71) Applicants: UNIVERSITY-INDUSTRY COOPERATION GROUP OF KYUNG HEE UNIVERSITY, Yongin-si, Gyeonggi-do (KR); CATHOLIC KWANDONG UNIVERSITY INDUSTRY FOUNDATION, Gangneung-si, Gangwon-do (KR)

(72) Inventors: Ik-Hyun CHO, Seoul (KR); Gyun Jee SONG, Incheon (KR)

(73) Assignees: UNIVERSITY-INDUSTRY COOPERATION GROUP OF KYUNG HEE UNIVERSITY, Yongin-si, Gyeonggi-do (KR); CATHOLIC KWANDONG UNIVERSITY INDUSTRY FOUNDATION, Gangneung-si, Gangwon-do (KR)

(21) Appl. No.: 18/845,647

(22) PCT Filed: Feb. 27, 2023
(86) PCT No.: PCT/KR2023/002730

§ 371 Date: Sep. 10, 2024

(30) Foreign Application Priority Data
Mar. 11, 2022 (KR) 10-2022-0030501

(51) Int. Cl. A61K 36/83; A61P 25/28

(52) U.S. Cl. CPC A61K 36/83; A61P 25/28; A61K 2236/33

(57) ABSTRACT

The present disclosure relates to a composition for prevention or treatment of neuroinflammation, comprising a Daphne genkwa flower bud extract as an active ingredient. According to the present disclosure, the Daphne genkwa flower bud extract of the present disclosure exhibits anti-inflammatory activity in nerve cells and thus has an effect of inhibiting neuroinflammation, inhibits (hyper) activation of microglia, and prevents neuronal loss and promotes the proliferation of nerve cells, thereby protecting nerves, and thus may be used for the use of reducing neuroinflammatory disease, neurodegenerative disease, or microglial activation.

### TECHNICAL FIELD

The present disclosure relates to a composition for prevention or treatment of neuroinflammation, containing a Daphne genkwa flower bud extract as an active ingredient.

### BACKGROUND ART

Neurodegenerative disease is a disease in which mental functions deteriorate due to the gradual structural and functional loss of neurons. The neurodegenerative disease is accompanied by symptoms such as dementia, extrapyramidal abnormalities, cerebellar abnormalities, sensory disorders, and motor disorders, as degeneration of nerve cells in specific parts of the nervous system progresses, and may also cause complex symptoms as abnormalities may occur in multiple areas at the same time. In this regard, the disease is diagnosed according to clinical features shown in a patient, but the symptoms variously occur and different diseases often show common clinical symptoms to make diagnosis difficult. The neurodegenerative disease shows signs of the disease gradually and often develops with aging. Once the disease develops, the disease continues to progress for several years or decades until death, and fundamental treatment is difficult, resulting in a very large social burden, and the cause of the disease is a genetic effect due to family history, but acquired factors are also known to play an important role therein. Depending on the clinical symptoms, the neurodegenerative disease is broadly classified into progressive dementia (Alzheimer's disease, etc.), neurological abnormalities (Pick's disease, etc.), posture and motor abnormalities (Parkinson's disease, etc.), progressive ataxia, muscle atrophy and weakness, sensory and motor disorders, etc. Among these, Alzheimer's disease, which is neurodegenerative disease with the highest prevalence rate of 6.54% in those aged 65 or older, accounts for 71.3% of all dementia cases, and cytotoxicity caused by β-amyloid plaques, neuroinflammation, and neurofibrillary tangles is drawing attention as a direct cause of disease.

The microglia are cells that perform a primary immune function in the central nervous system (CNS), and maintain a shape having thin and long branches and thin cell bodies and then change the shape into an activated shape having thick and short branches and round cell bodies to protect the neurons from these toxins which are introduced from the outside or produced therein. The activated microglia, unlike normal microglia, activate phagocytosis and proliferate cells, and express genes of cytokines such as TNF-α, IL-1β and IL-6, chemokines, inducible nitric oxide synthase (iNOS), cyclooxygenase-2 (COX-2), etc. to produce inflammatory mediators. The activation of microglia has an aspect of removing damaged cells and protecting nerve cells from bacteria or viruses invading from outside, but nitric oxide produced by iNOS with excessively increased expression, prostaglandins produced by COX-2, TNF-α, etc. are also toxic to nerve cells, and as a result, the activation of microglia worsens the injury to nerve cells. In addition, since substances released by dying nerve cells induce the microglial activation again, neurodegeneration is caught in a continuous vicious cycle. Actually, it has been reported that the microglial activation is associated with various neurodegenerative diseases, such as Alzheimer's disease, Parkinson's disease, Huntington's disease, Lou Gehrig's disease, Creutzfelt-Jakob disease (CJD), and multiple sclerosis. Activating substances of microglia include lipopolysaccharide (LPS) which is a bacterial endotoxin, interferon-γ, β-amyloid, ganglioside, etc. Signaling pathways involved in the activation of microglia include MAPK, PKC, ROS, NF-κB, etc. The mitogen-activated protein (MAP) kinase family is a key protein that acts as a signaling mediator in a cell, and is activated in response to various extracellular signals in the human body, such as inflammatory responses, apoptosis, cell differentiation, and growth, and activates transcription factors to regulate the transcription of necessary genes. The promoters of iNOS, TNF-α, and COX-2 genes expressed in the activated microglia commonly have an NF-κB binding region, and the expression of these genes is regulated by the activation of NF-κB. It is known that β-amyloid and LPS activate NF-κB in microglia, and gangliosides and thrombin also activate NF-κB in microglia. The activation of NF-κB by these activation substances occurs within 15 minutes and promotes the production of inflammatory cytokines. Although a relationship between the activation of microglia and neurodegenerative disease has not yet been fully found, it is generally accepted that the activation of microglia is related to the onset and progression of neurodegenerative disease. Therefore, the inhibiting of the activation of microglia will be an effective treatment to alleviate the progression of neurodegenerative disease.

Accordingly, the present inventors confirmed a method of inhibiting neuroinflammation using a natural extract through an experiment, because excessive activation of microglia and astrocytes, which cause neuroinflammation, causes neural injury and memory deterioration, and completed the present disclosure.

### DISCLOSURE

### Technical Problem

An object of the present disclosure is to provide a pharmaceutical composition for prevention or treatment of neuroinflammatory disease.

Another object of the present disclosure is to provide a pharmaceutical composition for prevention or treatment of neurodegenerative disease.

Yet another object of the present disclosure is to provide a pharmaceutical composition for reducing the activity of microglia in neurodegenerative disease.

Yet another object of the present disclosure is to provide a food composition for improvement or prevention of neuroinflammatory disease.

Yet another object of the present disclosure is to provide a method for preventing or treating neuroinflammatory disease comprising administering to a subject a pharmaceutical composition for prevention 1 or treatment of neuroinflammatory disease comprising a Daphne genkwa flower bud extract in a pharmaceutically effective amount as an active ingredient.

### Technical Solution

In order to achieve the above aspects, the present disclosure provides a pharmaceutical composition for prevention or treatment of neuroinflammatory disease, comprising a Daphne genkwa flower bud extract as an active ingredient.

The present disclosure also provides a pharmaceutical composition for prevention or treatment of neurodegenerative disease, comprising a Daphne genkwa flower bud extract as an active ingredient.

The present disclosure also provides a pharmaceutical composition for reducing the activity of microglia in neurodegenerative disease, comprising a Daphne genkwa flower bud extract as an active ingredient.

The present disclosure also provides a food composition for improvement or prevention of neuroinflammatory disease, comprising a Daphne genkwa flower bud extract as an active ingredient.

The present disclosure also provides a method for preventing or treating neuroinflammatory disease comprising administering to a subject a pharmaceutical composition for prevention or treatment of neuroinflammatory disease comprising a Daphne genkwa flower bud extract in a pharmaceutically effective amount as an active ingredient.

### Advantageous Effects

According to the present disclosure, the Daphne genkwa flower bud extract of the present disclosure exhibits anti-inflammatory activity in nerve cells and thus has an effect of inhibiting neuroinflammation, inhibits (over) activation of microglia, and prevents neuronal loss and promotes the proliferation of nerve cells, thereby protecting nerves, and thus can be used for the use of reducing neuroinflammatory disease, neurodegenerative disease, or microglial activation.

### DESCRIPTION OF DRAWINGS

FIG. 1 is a diagram illustrating an anti-inflammatory effect of a Daphne genkwa flower bud extract (GFE) on microglia:
A: NO production and IC₅₀ of HAPI cells treated with LPS and/or GFE;
B: Cell viability of HAPI cells 24 hours after stimulation with LPS and/or GFE;
C: NO production in BV-2 cells treated with LPS and/or GFE;
D: Cell viability of BV-2 cells 24 hours after stimulation with LPS and/or GFE;
E: NO production of MGCs treated with LPS and/or GFE; and
F: Cell viability of MGCs 48 hours after stimulation with LPS and/or GFE.

FIG. 2 is a diagram illustrating a pro-inflammatory inhibitory effect of GFE in MGCs:
A: Expression levels of mRNAs of TNF-α and iNOS after 48 hours of treatment with LPS (1 μg/ml) and/or GFE (10 μg/mL);
B: Quantitative graph for mRNA of TNF-α;
C: Quantitative graph for mRNA of iNOS; and
D: Extracellular release levels of TNF-α protein in MGC cell culture medium after 18 hours of treatment with LPS and/or GFE.

FIG. 3 is a diagram illustrating an inhibitory effect of GFE on microglia hyperactivation in a mouse brain:
A: Administration schedule of LPS (4 mg/kg) and GFE (50, 100, and 200 mg/kg);
B: Results of Iba-1 immunostaining in mouse brains in a control group, an LPS (4 mg/kg) administered group and an LPS (4 mg/kg)+GFE (200 mg/kg) administered group (Iba-1+single microglia);
C: Relative fluorescence intensity for Iba-1 in mouse brains in a control group, an LPS (4 mg/kg) administered group and an LPS (4 mg/kg)+GFE (200 mg/kg) administered group;
D: Areas of Iba-1+ cells in mouse brains in a control group, an LPS (4 mg/kg) administered group and an LPS (4 mg/kg)+GFE (200 mg/kg) administered group; E: The numbers of Iba-1+ cells in mouse brains in a control group, an LPS (4 mg/kg) administered group and an LPS (4 mg/kg)+GFE (200 mg/kg) administered group; and
F: Quantitative graph of mRNA levels of IL-1b in mouse brains in a control group, an LPS (4 mg/kg) administered group, a GFE (200 μg/ml) administered group, and an LPS (4 mg/kg)+GFE (50, 100 and 200 μg/ml) administered group.

FIG. 4 is a diagram confirming a neuroprotective effect of GFE in mouse brain tissue and primary cortical nerve cells:
A: Immunostaining for NeuN in thalamic sections of the mouse brain cortical regions in a vehicle (control) group, an LPS (4 mg/kg) administered group, and an LPS (4 mg/kg)+GFE (200 mg/kg) administered group;
B: Quantitative graph of the relative number of NeuN-positive cells per field; and
C: Quantitative graph of proliferation of primary cortical nerve cells based on CCK8 assay in mouse fetal cortical nerve cells treated with conditioned medium derived from HAPI cells treated with LPS (100 ng/ml) and/or GFE (10 μg/ml) for 48 hours.

FIG. 5 is a diagram illustrating a neuroprotective microglia function promotion effect of GFE:
A: mRNA levels of Arg1 in MGCs treated or not with GFE (10 μg/ml) for 48 h;
B: mRNA levels of BDNF in MGCs treated or not with GFE (10 μg/ml) for 48 h; C: Immunofluorescence staining of primary microglia treated with GFE (10 μg/ml) or vehicle (0.1% DMSO) for 48 h; and
D: Number of zymosan particles per cell.

FIG. 6 is a diagram illustrating a signaling pathway related to an anti-neuroinflammatory effect of GFE.

FIG. 7 is a diagram illustrating a summary of the effects of GFE of the present invention.

### BEST MODE OF THE INVENTION

Hereinafter, embodiments of the present disclosure will be described in detail with reference to the accompanying drawings. However, the following embodiments are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

Terminologies used herein are terminologies used to properly express preferred embodiments of the present disclosure, which may vary according to a user, an operator's intention, or customs in the art to which the present disclosure pertains. Therefore, these terminologies used herein will be defined based on the contents throughout the specification. Throughout the specification, unless explicitly described to the contrary, when a certain part “comprises” a certain component, it will be understood to imply the inclusion of stated elements but not the exclusion of any other elements.

In one aspect, the present disclosure relates to a pharmaceutical composition for prevention or treatment of neuroinflammation, comprising a Daphne genkwa flower bud extract as an active ingredient.

In an embodiment, the extract may be extracted with at least one solvent selected from the group consisting of water, organic solvents, subcritical fluids, and supercritical fluids. The organic solvent may be any one selected from the group consisting of lower alcohols having 1 to 4 carbon atoms, hexane (n-hexane), ether, glycerol, propylene glycol, butylene glycol, ethyl acetate, methyl acetate, dichloromethane, chloroform, ethyl acetate, acetone, methylene chloride, cyclohexane, petroleum ether, benzene and mixed solvents thereof, and most preferably methanol.

In an embodiment, the neuroinflammation may be brain neuroinflammation.

In an embodiment, the neuroinflammatory disease may be neuroinflammatory disease with increased activity of microglia or astrocytes.

In an embodiment, the Daphne genkwa flower bud extract may inhibit neuroinflammation in the cerebral cortex induced by the activity of microglia or astrocytes.

As used herein, the term “extract” refers to an active ingredient isolated from a natural product, that is, a substance showing a desired activity. The extract may be obtained through an extraction process using water, an organic solvent, or a mixed solvent thereof, and includes extract, dry powders of the extract, or all forms formulated using the dry powders. In addition, the extract includes fractions obtained from the extract subjected to the extraction process. The extraction method of the extract is not particularly limited, and may be extracted by, for example, stirring extraction, shaking extraction, hot water extraction, cold immersion extraction, reflux cooling extraction, or ultrasonic extraction. The extraction solvent may be a polar solvent such as water, and lower alcohols having C₁-C₄, non-polar solvents such as hexane, chloroform, dichloromethane or ethyl acetate, or mixtures of two or more of these.

The composition of the present disclosure may be prepared as a pharmaceutical composition for prevention or treatment of neuroinflammatory disease by additionally containing not only a Daphne genkwa flower bud extract, but also other active ingredients having the same or similar function, or by additionally containing other active ingredients having different functions from the ingredients.

Microglia, which act as macrophages in the brain, are important cells that regulate immune responses in the central nervous system (CNS). The activation plays an important role in maintaining homeostasis of the CNS by removing foreign substances caused by drugs or toxins and secreting nerve growth factors. However, when exposed to harmful stresses such as signals generated from damaged neurons, accumulation of abnormally shaped proteins mutated by external stimuli, and invasion of pathogens, the activity of microglia may increase excessively to cause damage to nerve cells, thereby causing neurodegenerative diseases. That is, unlike normal microglia, the excessively activated microglia activate phagocytosis, proliferate cells, and express pro-inflammatory cytokines and inflammation-related genes to produce inflammatory mediators.

The activation of microglia has a positive effect of removing damaged cells and protecting nerve cells from bacteria or viruses invading from outside, but the activation of astrocytes, production of nitric oxide (NO), increased levels of cytokine of TNF-α, etc. are toxic even to nerve cells and lead to the death of nerve cells. As a result, the activation of microglia worsens the damage to nerve cells and causes neurodegenerative diseases. Therefore, a method for inhibiting excessive activity of microglia may be a method for treating neurodegenerative disease.

In addition, the astrocytes are also known to play an important role in maintaining normal brain activity, and particularly known to play a role in the formation of synapses in nerve cells, regulation of synapse numbers, synapse function, and differentiation of neural stem cells into neurons. However, when these astrocytes have an excessive response, that is, remain in an excessively activated state, the astrocytes activate microglia, cause the death of nerve cells, and induce the death of neighboring nerve cells, which may cause neurodegenerative diseases. Therefore, the inhibition of the activation of activated astrocytes may also be a new treatment method for neurodegenerative diseases.

In one aspect, the present disclosure relates to a pharmaceutical composition for prevention or treatment of neurodegenerative disease, comprising a Daphne genkwa flower bud extract as an active ingredient.

In an embodiment, the Daphne genkwa flower bud extract may be a Daphne genkwa flower bud methanol extract.

In an embodiment, the neurodegenerative disease may be any one selected from the group consisting of Alzheimer's disease, Parkinson's disease, Creutzfeldt-Jakob disease (CJD), Hallervorden-Spatz disease, Huntington's disease, multiple system atrophy, dementia, frontotemporal dementia, amyotrophic lateral sclerosis, spinal muscular atrophy, spinocerebellar atrophy (SCA), meningoencephalitis, bacterial meningoencephalitis, viral meningoencephalitis, CNS autoimmune disorder, multiple sclerosis (MS), and acute ischemic injury.

In an embodiment, the neurodegenerative disease may be neurodegenerative disease with increased activity of astrocytes or microglia.

In one aspect, the present disclosure relates to a pharmaceutical composition for reducing the activity of microglia in neurodegenerative disease, comprising a Daphne genkwa flower bud extract as an active ingredient.

In an embodiment, the composition of the present disclosure may inhibit hyperactivation of microglia in the cerebral cortex.

In an embodiment, the composition of the present disclosure may exhibit a protective effect against damage caused by activated microglia to nerve cells.

In an embodiment, the composition of the present disclosure may inhibit the expression of proinflammatory cytokines and inducible nitric oxide synthase (iNOS).

In an embodiment, the composition of the present disclosure may inhibit the production of nitric oxide (NO).

In an embodiment, the composition of the present disclosure may reduce the expression and release of IL-1β in the cerebral cortex.

As used herein, the term “prevention” means any action of inhibiting or delaying the occurrence, spread, and recurrence of neuroinflammatory disease or neurodegenerative disease by administering the pharmaceutical composition according to the present disclosure. The “treatment” means any action that improves or beneficially changes the symptoms of neuroinflammatory disease or neurodegenerative disease by administering the composition of the present disclosure. Those skilled in the art to which the present disclosure pertains will be able to determine the degree of improvement, enhancement and treatment by knowing the exact criteria of disease for which the composition of the present disclosure is effective by referring to data presented by the Korean Academy of Medical Sciences, etc.

As used herein, the term “therapeutically effective amount” used in combination with the active ingredient means an effective amount to prevent or treat neuroinflammatory disease or neurodegenerative disease, and the therapeutically effective amount of the composition of the present disclosure may vary depending on many factors, such as a method of administration, a target site, the condition of a patient, and the like. Accordingly, when used in the human body, a dose should be determined as an appropriate amount in consideration of both safety and efficiency. It is also possible to estimate the amount used in humans from the effective amount determined through animal experiments. These matters to be considered when determining the effective amount are described in, for example, Hardman and Limbird, eds., Goodman and Gilman's The Pharmacological Basis of Therapeutics, 10th ed. (2001), Pergamon Press; and E. W. Martin ed., Remington's Pharmaceutical Sciences, 18th ed. (1990), Mack Publishing Co.

The pharmaceutical composition of the present disclosure is administered in a pharmaceutically effective amount. As used herein, the term “pharmaceutically effective amount” refers to an amount enough to treat diseases at a reasonable benefit/risk ratio applicable to medical treatment and enough not to cause side effects. The effective dose level may be determined according to factors including a health condition of a patient, a type of neuroinflammatory disease or neurodegenerative disease, an occurrence cause of neuroinflammatory disease or neurodegenerative disease, severity, drug activity, sensitivity to a drug, an administration method, an administration time, an administration route and an excretion rate, a treatment period, and drugs used in combination or concurrently, and other factors well-known in medical fields. The composition of the present disclosure may be administered as an individual therapeutic agent or in combination with other therapeutic agents, and may be administered sequentially or simultaneously with conventional therapeutic agents, and may be administered singly or multiply. It is important to administer an amount capable of obtaining a maximum effect with a minimal amount without side effects by considering all the factors, which may be easily determined by those skilled in the art.

The pharmaceutical composition of the present disclosure may include a carrier, a diluent, an excipient, or a combination of two or more thereof, which are commonly used in biological agents. As used herein, the term “pharmaceutically acceptable” means exhibiting non-toxic properties to cells or humans exposed to the composition. The carrier is not particularly limited as long as the carrier is suitable for in vivo delivery of the composition, and may be used by combining, for example, compounds described in Merck Index, 13th ed., Merck & Co. Inc., saline, sterile water, a Ringer's solution, buffered saline, a dextrose solution, a maltodextrin solution, glycerol, ethanol, and one or more of these components, and if necessary, other conventional additives such as an antioxidant, a buffer, and a bacteriostat may be added. In addition, the pharmaceutical composition may be prepared in injectable formulations such as an aqueous solution, a suspension, and an emulsion, pills, capsules, granules, or tablets by further adding a diluent, a dispersant, a surfactant, a binder, and a lubricant. Furthermore, the pharmaceutical composition may be prepared preferably according to each disease or ingredient using a suitable method in the art or a method disclosed in Remington's Pharmaceutical Science (Mack Publishing Company, Easton PA, 18th, 1990).

In an embodiment, the pharmaceutical composition may be one or more formulations selected from the group including oral formulations, external formulations, suppositories, sterile injection solutions and sprays, and more preferably oral or injectable formulations.

As used herein, the term “administration” means providing a predetermined substance to a subject or patient by any suitable method, and the pharmaceutical composition may be administered parenterally (e.g., applied as intravenously, subcutaneously, intraperitoneally or topically injectable formulations) or orally according to a desired method. The dose range may vary depending on the body weight, age, sex, and health condition of a patient, a diet, an administration time, an administration method, an excretion rate, the severity of a disease, etc. Liquid formulations for oral administration of the composition of the present disclosure correspond to suspensions, internal solutions, emulsions, syrups, etc., and may include various excipients, such as wetting agents, sweeteners, fragrances, preservatives, and the like, in addition to water and liquid paraffin, which are commonly used simple diluents. Formulations for parenteral administration include sterilized aqueous solutions, non-aqueous solvents, suspensions, emulsions, lyophilized agents, suppositories, and the like. The pharmaceutical composition of the present disclosure may also be administered by any device capable of migrating the active substance to a target cell. Preferred administration methods and formulations are intravenous injections, subcutaneous injections, intradermal injections, intramuscular injections, drop injections, etc. The injections may be prepared by using aqueous solvents such as a physiological saline solution and a ringer solution, and non-aqueous solvents such as vegetable oils, higher fatty acid esters (e.g., ethyl oleate), and alcohols (e.g., ethanol, benzyl alcohol, propylene glycol, or glycerin). The injections may include pharmaceutical carriers, such as a stabilizer for the prevention of degeneration (e.g., ascorbic acid, sodium hydrogen sulfite, sodium pyrosulfite, BHA, tocopherol, EDTA, etc.), an emulsifier, a buffer for pH control, and a preservative to inhibit microbial growth (e.g., phenyl mercury nitrate, thimerosal, benzalkonium chloride, phenol, cresol, benzyl alcohol, etc.).

As used herein, the term “subject” refers to all animals including monkeys, cows, horses, sheep, pigs, chickens, turkeys, quails, cats, dogs, mice, rats, rabbits or guinea pigs including humans who have developed or may develop the neuroinflammatory disease or neurodegenerative disease, and the pharmaceutical composition of the present disclosure may be administered to the subject to effectively prevent or treat the diseases. The pharmaceutical composition of the present disclosure may be administered in conjunction with conventional therapeutic agents.

The pharmaceutical composition of the present disclosure may further include a pharmaceutically acceptable additive. At this time, the pharmaceutically acceptable additive may be used with starch, gelatinized starch, microcrystalline cellulose, lactose, povidone, colloidal silicon dioxide, calcium hydrogen phosphate, lactose, mannitol, syrup, Arabic gum, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, white sugar, dextrose, sorbitol, talc, and the like. The pharmaceutically acceptable additive according to the present disclosure is preferably included in an amount of 0.1 part by weight to 90 parts by weight based on the composition, but is not limited thereto.

In one aspect, the present disclosure relates to a food composition for improvement or prevention of neuroinflammatory disease, containing a Daphne genkwa flower bud extract as an active ingredient.

In an embodiment, the extract may be a methanol extract.

When the composition of the present disclosure is used as the food composition, the composition may be added as it is or used with other foods or food ingredients, and may be appropriately used according to a general method. The composition may include food acceptable supplement additives in addition to the active ingredients, and the mixing amount of the active ingredients may be appropriately determined depending on the purpose of use (prevention, health or therapeutic treatment).

As used herein, the term “food supplement additive” means a component that may be supplementally added to food, and may be appropriately selected and used by those skilled in the art as being added to prepare a health functional food of each formulation. Examples of the food supplement additive include various nutrients, vitamins, minerals (electrolytes), flavors such as synthetic and natural flavors, colorants and fillers, pectic acid and salts thereof, alginic acid and salts thereof, organic acids, protective colloidal thickeners, pH adjusters, stabilizers, preservatives, glycerin, alcohols, carbonation agents used in carbonated drinks, and the like, but the types of food supplement additive of the present disclosure are not limited by the examples.

The food composition of the present disclosure may include a health functional food. As used herein, the term “health functional food” refers to food prepared and processed in the form of tablets, capsules, powders, granules, liquids and pills by using raw materials or ingredients having functionalities useful to the human body. Here, the ‘functionality’ means regulating nutrients to the structure and function of the human body or obtaining effects useful for health applications such as physiological action. The health functional food of the present disclosure is able to be prepared by methods to be commonly used in the art and may be prepared by adding raw materials and ingredients which are commonly added in the art in preparation. In addition, the formulations of the health functional food may also be prepared with any formulation recognized as a health functional food without limitation. The food composition of the present disclosure may be prepared in various types of formulations, and unlike general drugs, the food composition has an advantage that there is no side effect that may occur when taking a drug in a long-term due to using the food as a raw material, and has excellent portability, and the health functional food of the present disclosure can be taken as supplements to enhance the effects of agents for prevention or treatment of neuroinflammatory disease or neurodegenerative disease.

In addition, there is no limitation in types of health food in which the composition of the present disclosure may be used. In addition, the composition comprising Daphne genkwa flower bud extract of the present disclosure as the active ingredient may be prepared by mixing known additives with other suitable auxiliary ingredients that may be contained in the health functional food according to the selection of those skilled in the art. Examples of food to be added include meat, sausage, bread, chocolate, candy, snacks, confectionery, pizza, ramen, other noodles, gum, dairy products including ice cream, various soups, beverages, tea, drinks, alcoholic beverages, vitamin complexes and the like, and may be prepared to be added to extract, tea, jelly, juice, and the like prepared by using the extract according to the present disclosure as a main ingredient.

In addition, the present disclosure provides a method for preventing and treating neuroinflammatory disease, including administering the Daphne genkwa flower bud extract in a pharmaceutically effective amount to a subject. The pharmaceutical composition of the present disclosure is administered in a therapeutically effective amount or pharmaceutically effective amount. The term “pharmaceutically effective amount” refers to an amount enough to treat diseases at a reasonable benefit/risk ratio applicable to medical treatment, and the effective dose level may be determined according to factors including the type, severity, age, and sex of a subject, the activity of a drug, the sensitivity to a drug, a time of administration, a route of administration, an excretion rate, duration of treatment, and drugs to be simultaneously used, and other factors well-known in the medical field.

Hereinafter, the present disclosure will be described in more detail with reference to the following Examples. However, the following Examples are only intended to embody the contents of the present disclosure, and the present disclosure is not limited thereto.

### Modes of the Invention

### Example 1. Preparation of Daphne Genkwa Extract

The flower buds of Daphne genkwa were purified, dried, and ground to be prepared in a fine powder form, and the powder was refluxed twice using 70% methanol (3.5 L) (ratio of powder:methanol of 1:7 (w/w)). Thereafter, the mixture was filtered and concentrated under reduced pressure through a freeze-drying process, and 100 mg/ml of a Daphne genkwa flower bud extract (GFE) was prepared using dimethyl sulfoxide (DMSO) as a solvent.

### Example 2. Cytotoxicity and Anti-Inflammatory Activity of Daphne Genkwa Extract

In order to confirm the anti-inflammatory effect of the Daphne genkwa flower bud extract (GFE) prepared in Example 1 above on nerve cells, primary mixed glial cells (MGCs) and highly aggressively proliferating immortalized (HAPI) cells were cultured in a Dulbecco's modified eagle medium (DMEM) (Gibco, Grand Island, NY, USA) containing 10% FBS (Gibco) and 100 U/mL penicillin/streptomycin (Gibco) at 37° C. and 5% CO₂, and BV-2 cells were cultured in a medium containing 5% FBS and 50 μg/mL gentamicin at 37° C. and 5% CO₂. Thereafter, the cells were stimulated with LPS (HAPI and BV-2:100 ng/mL for 24 h, MGCs: 1 μg/mL for 48 h) in the presence or absence of GFE (200 to 5 μg/mL) to induce inflammatory responses, and then cell viability and NO production level were confirmed (Gupta et al., 2020). In addition, in the case of HAPI cells, a half-maximal inhibitory concentration (IC₅₀) of GFE was also evaluated, and an effect of GFE concentration on LPS-induced NO production was measured.

As a result, GFE did not exhibit cytotoxicity in HAPI cells (microglia cell line), and LPS-induced NO production was significantly inhibited by GFE, and the minimum concentration of GFE that inhibited LPS-induced NO production was 10 μg/ml (FIG. 1A and FIG. 1B). In addition, it was shown that GFE significantly inhibited LPS-induced NO production even in BV-2 cells, which were another microglia cell line (FIG. 1C and FIG. 1D). Moreover, even in primary MGCs, including microglia and astrocytes, which played a key role in neuroinflammation, it was shown that GFE significantly inhibited LPS-induced NO release without exhibiting cytotoxicity (FIG. 1E and FIG. 1F).

Through this, it was confirmed that the GFE of the present disclosure had an anti-inflammatory effect, particularly, a neuroinflammation inhibitory effect.

### Example 3. Anti-Inflammatory Mechanism of Daphne Genkwa Extract

To molecularly confirm the neuroinflammation inhibitory effect, primary MGCs were cultured for 48 hours and exposed to GFE (10 μg/ml) and LPS (1 μg/ml) for 24 hours. Then, the mRNA expression levels of proinflammatory cytokines TNF-α and inducible nitric oxide synthase (iNOS) were confirmed by RT-PCR, and the extracellular release of TNF-α protein was confirmed by ELISA using a cell culture medium.

As a result, the TNF-α mRNA level increased by LPS compared to a control group (vehicle) was reduced to a level similar to the control group in a GFE treated group (FIG. 2A and FIG. 2B), and the iNOS mRNA level increased by LPS was also significantly inhibited by GFE (FIG. 2A and FIG. 2C). In addition, the increase in TNF-α protein release induced by LPS was significantly inhibited by GFE treatment (FIG. 2D).

Through this, it was confirmed that the GFE had an anti-inflammatory effect on microglia.

### Example 4. Inhibitory Effect of Daphne Genkwa Extract on In Vivo Microglia Hyperactivation

The effect of the Daphne genkwa extract on microglial activation was confirmed using an LPS-induced neuroinflammation mouse model. Specifically, 8-to 9-week-old C57BL/6J female mice (Narabiotec Co., Ltd., Seoul, Republic of Korea) were administered with GFE (50, 100, and 200 mg/kg) by oral gavage for 3 days, and injected intraperitoneally with LPS (4 mg/kg) 1 hour after oral administration on days 1 and 3 (FIG. 3A). 72 h after LPS injection, the mice were euthanized under isoflurane (1.5% to 2.0%) anesthesia and intracardially perfused with 0.9% saline and 4% paraformaldehyde (in 0.1 M PBS, pH 7.4). The brains were extracted, post-fixed in 4% paraformaldehyde for 24 hours, and then cryopreserved sequentially in 10%, 20%, and 30% sucrose (in PBS) at 4° C. Subsequently, 20-μm-thick serial sagittal sections were obtained using a CM3050S freezing microtome (Leica, Wetzlar, Germany), and immunofluorescent staining for ionized calcium binding adaptor molecule 1 (Iba1) was performed in the sections according to a method described by Gupta et al., 2020. In addition, in order to evaluate the concentration-dependent effect of GFE on IL-1B cytokine release after LPS treatment, the IL-1b mRNA level in the mouse brain was identified by qRT-PCR.

As a result, it was shown that the immunoreactive level of Iba-1, a molecular marker of microglia, in the cortical area of the mouse brain was significantly enhanced in an LPS injection group compared to the control group (FIG. 3B and FIG. 3C). In addition, it was shown that the number of Iba-1-positive microglia and an Iba1+ cell area were increased in the LPS-administered group compared to the control group (FIG. 3D and FIG. 3E). It was shown that these morphological characteristics and increased Iba-1 immunoreactivity of microglia in the cerebral cortex were significantly inhibited in mice administered with GFE, and in the mouse brain administered with GFE, small and round somas with many ramified microglia were present, as in the brain tissue of the control mouse (FIG. 3B to FIG. 3E). In addition, it was shown that LPS-induced IL-1β release in the brain cortical tissue was significantly reduced in a dose-dependent manner by GFE treatment (FIG. 3F).

### Example 5. Effects of Daphne Genkwa Extract on Preventing In Vivo Neuronal Loss and Promoting Proliferation of Nerve Cells

In Example, since GFE improved neuroinflammation in the mouse brain, in order to confirm whether GFE protected neurons in an LPS-injected mouse brain tissue, the brain sections of the mouse in Example 4 were analyzed by immunostaining using a neural marker NeuN to determine the number of neurons in the prefrontal cortex. In addition, in order to confirm this even at the cellular level, the mouse brain at the age of 2 to 3 days was extracted, and the cortical region of the brain was isolated, cut into small pieces, and digested in 0.025% trypsin/ethylene-diamine-tetraacetic acid for 30 minutes. Thereafter, the tissue was pulverized to obtain single cells (primary cortical nerve cells). The primary cortical nerve cells were seeded onto a poly-D-lysine-coated cell culture dish and cultured in a Neurobasal medium containing glutamine (2 mM) and 1% penicillin/streptomycin, and treated with a medium of HAPI cells treated with LPS (100 ng/ml) in the presence or absence of GFE (10 μg/ml) for 48 h. Thereafter, for cell proliferation analysis, 10 μl of a CCK-8 solution from cell counting kit-8 (CCK8) (Dojindo Molecular Technologies, Inc., Rockville, MD, USA) was added to a cell suspension (100 μl/well) in a 96-well plate, and then the absorbance was measured at 450 nm using a microplate reader after 2 hours.

Immunostaining analysis results showed that NeuN-positive cells were significantly reduced in the LPS-administered group compared to the control group, but the loss of NeuN-positive nerve cells was significantly inhibited in the LPS and GFE-administered groups (FIG. 4A and FIG. 4B). In addition, as a result of the proliferation analysis of the primary cortical nerve cells, it was shown that the number of primary cortical nerve cells was significantly increased in the GFE treated group compared to the control group (FIG. 4C).

Through this, it was confirmed that the GFE promoted the neuroprotective effect.

### Example 6. Neuroprotective Effect of Daphne Genkwa Extract

To confirm the neuroprotective effect of (alternatively activated) microglia of GFE, the mRNA levels of Arg1 as an alternatively activated microglia marker, and brain-derived neurotrophic factor (BDNF) as a neurotrophic factor were confirmed by qRT-PCR in MGCs treated or not with GFE (10 μg/ml) for 48 hours. In addition, in order to confirm the phagocytic activity of microglia, primary microglia treated or not with GFE (10 μg/ml) for 48 hours were treated with Zymosan-Red particles (10 g/ml), and the number of zymosan particles/cell was quantified in 160 control cells (vehicle, 0.1% DMSO) and 130 GFE-treated cells.

As a result, it was shown that the mRNA level of Arg1 was significantly increased in GFE-treated MGCs compared to the control group (FIG. 5A), and the mRNA level of BDNF was also significantly increased in the GFE-treated group (FIG. 5B). In addition, it was shown that the number of labeled zymosan particles in primary microglia treated with GFE was significantly increased compared to the control group (FIG. 5C and FIG. 5D).

Through this, it was confirmed that the microglial activation after GFE treatment induced the neuroprotective function of microglia.

### Example 7. Anti-Neuroinflammatory Effect Mechanism of Daphne Genkwa Extract

To identify a signaling pathway related with the microglial activation effect of GFE, MGCs were pretreated with a MAPK inhibitor PD98059 (10 μM), a ULK inhibitor SBI-0206965 (5 μM), and an NF-κ inhibitor Bay 11-7082 (2.5 μM) for 1 h, and then stimulated with LPS (1 μg/mL) in the presence/absence of GFE (10 μg/mL) for 48 h, and NO production was identified.

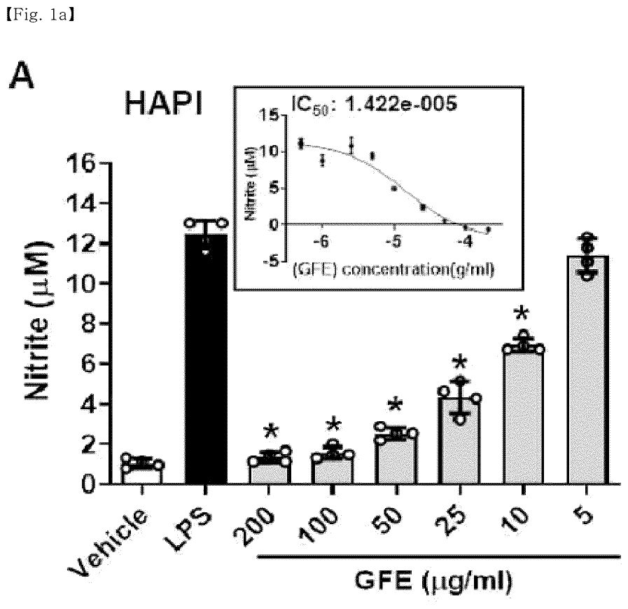

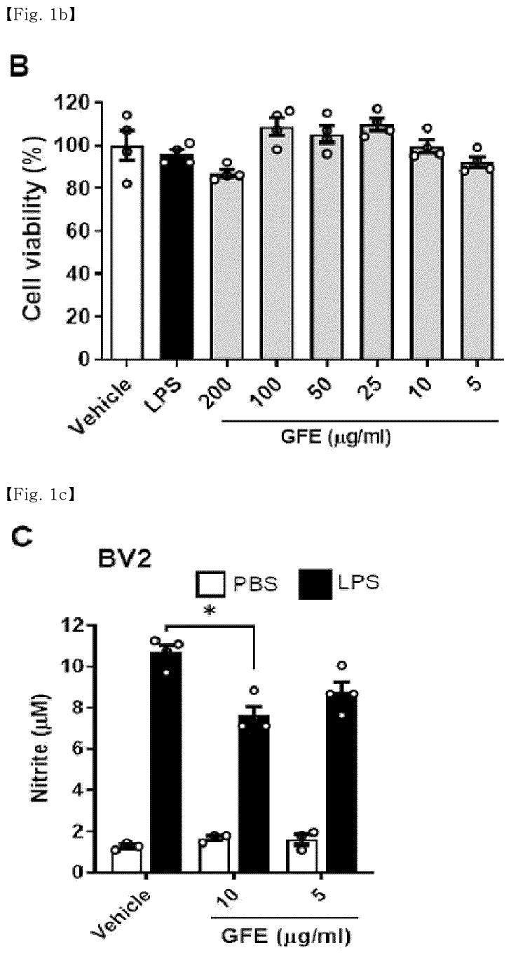

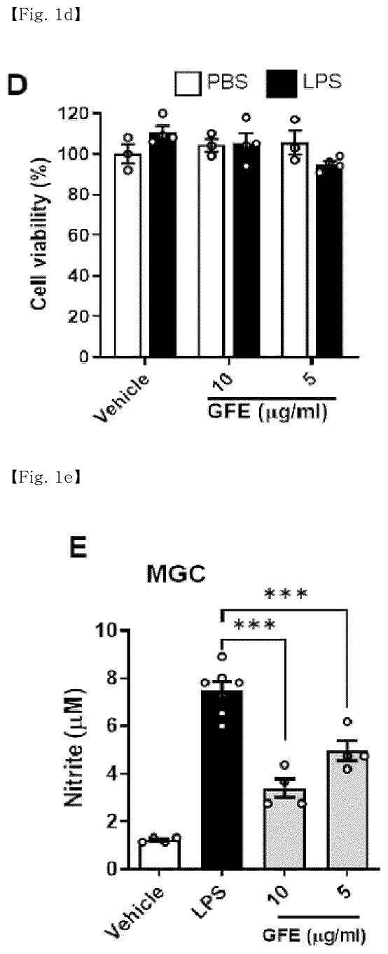

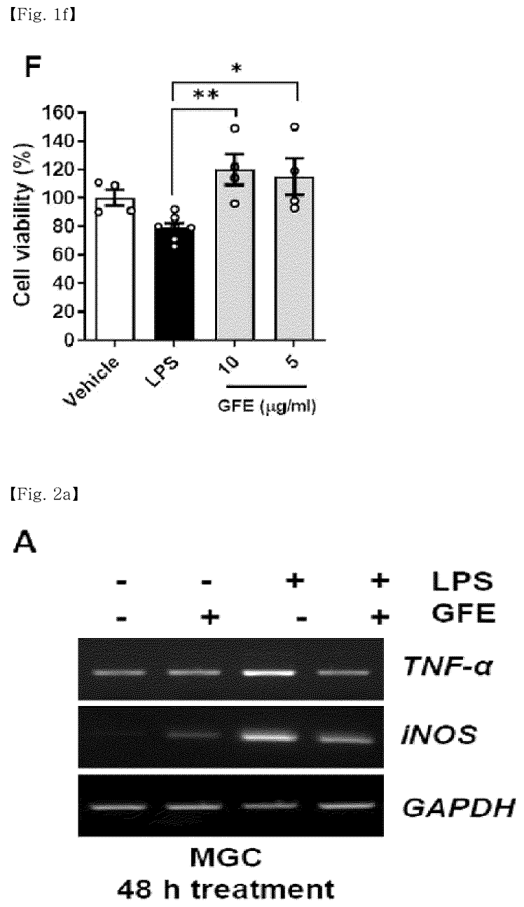

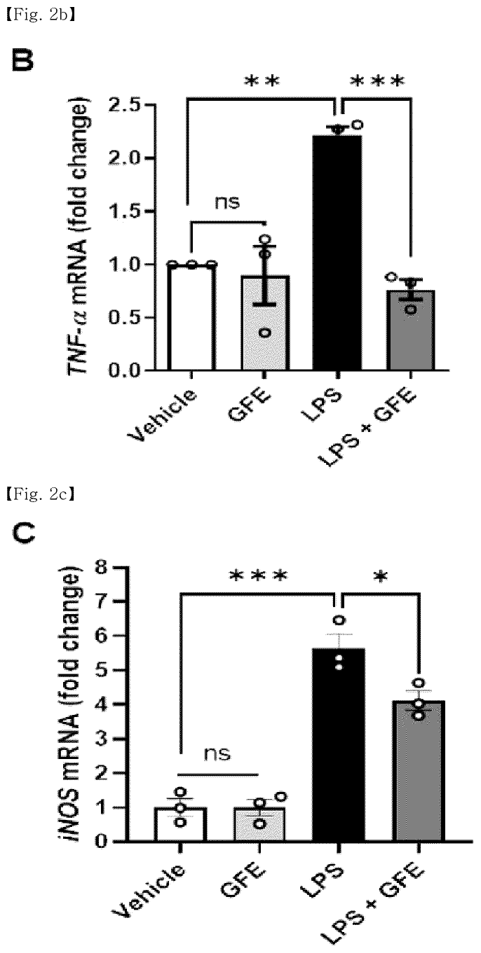

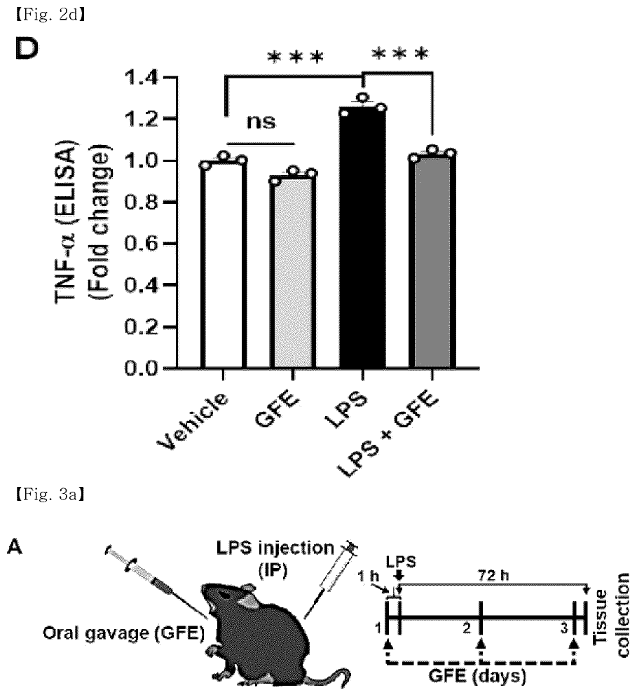

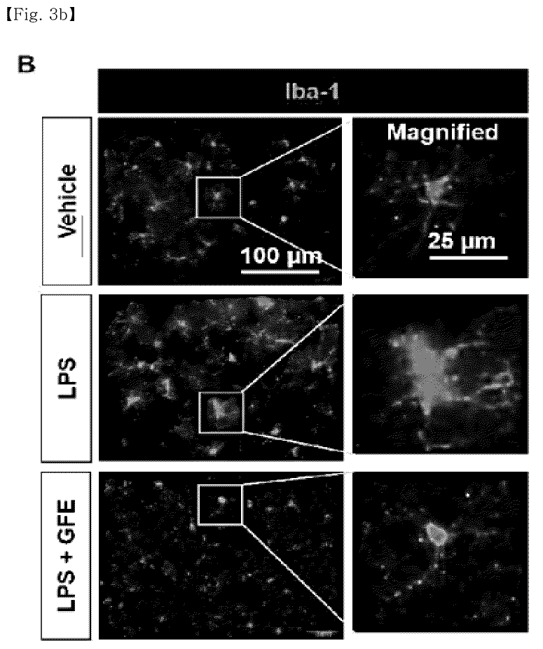

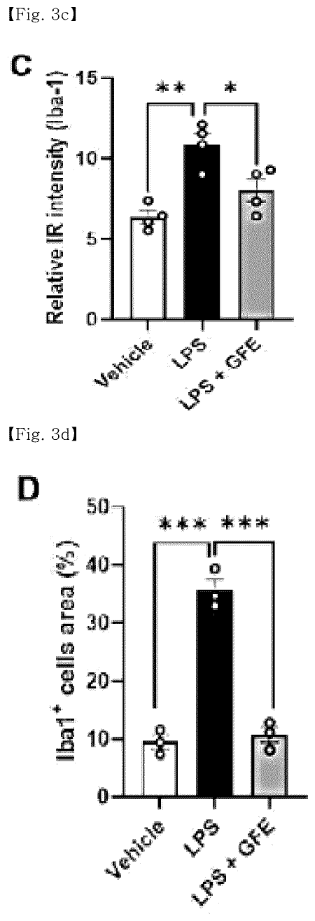

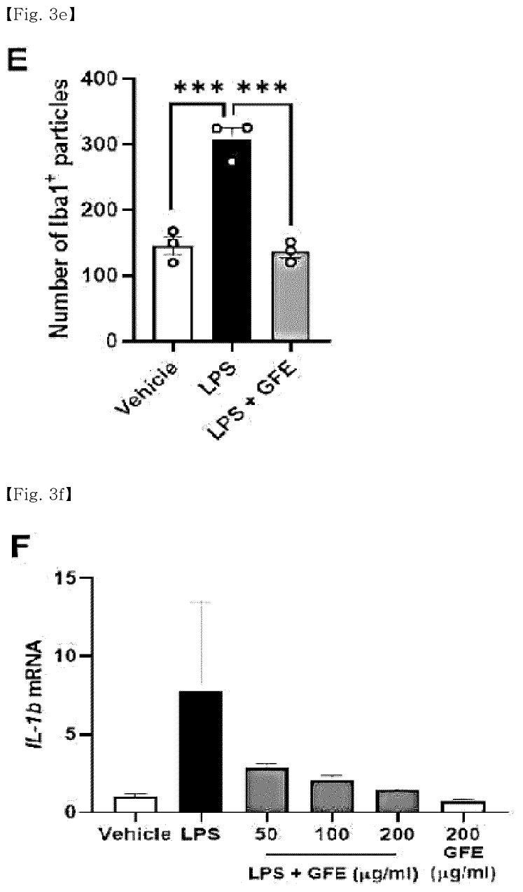

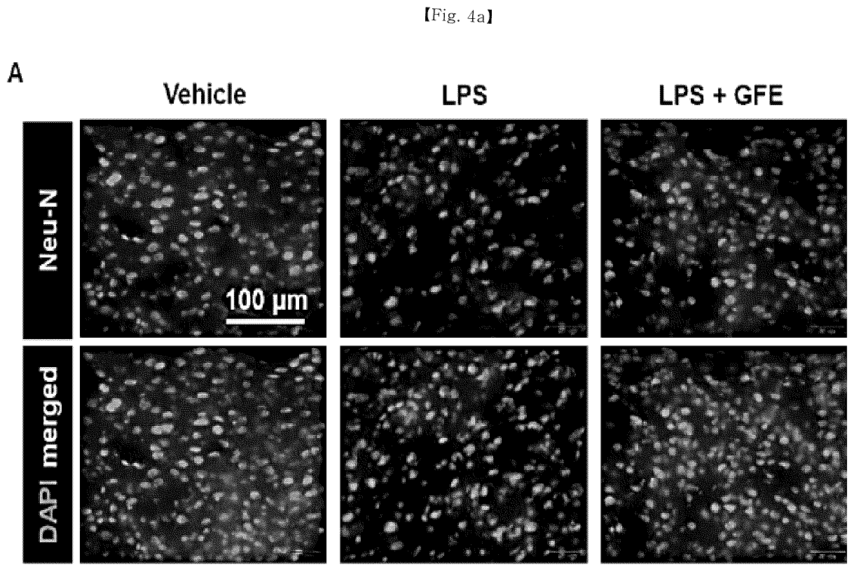

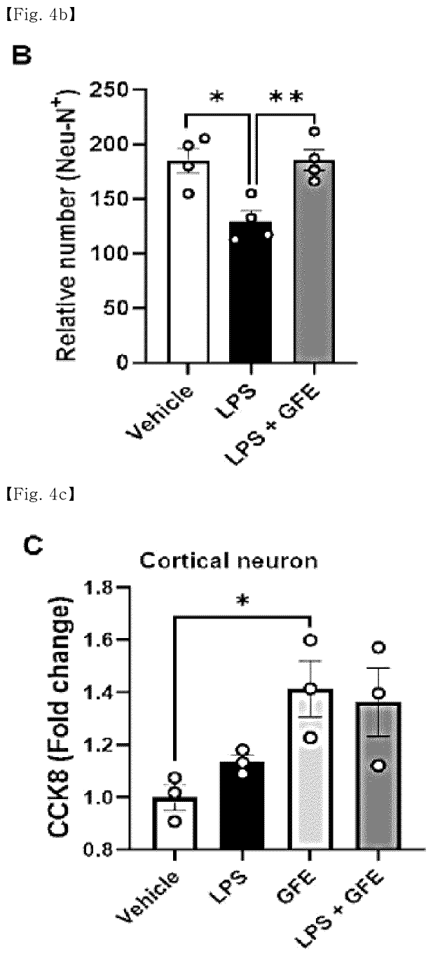

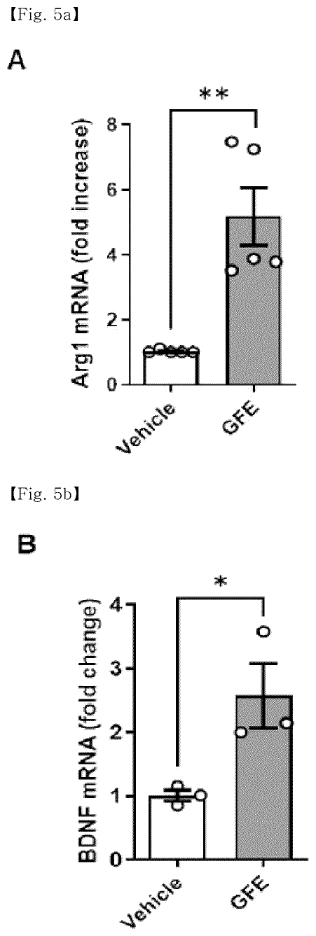

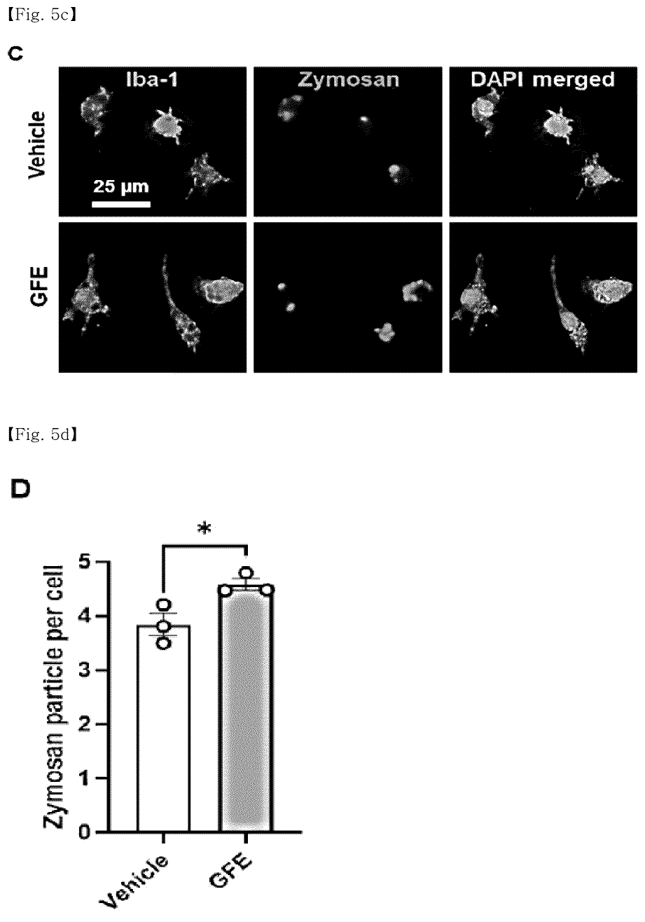

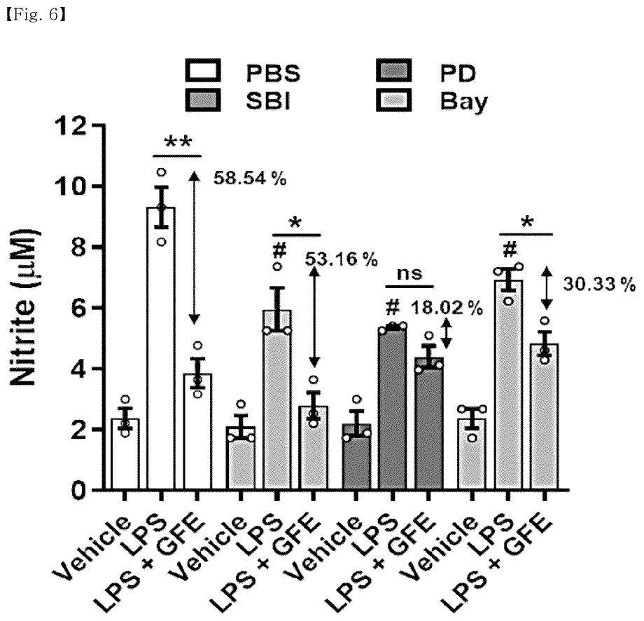

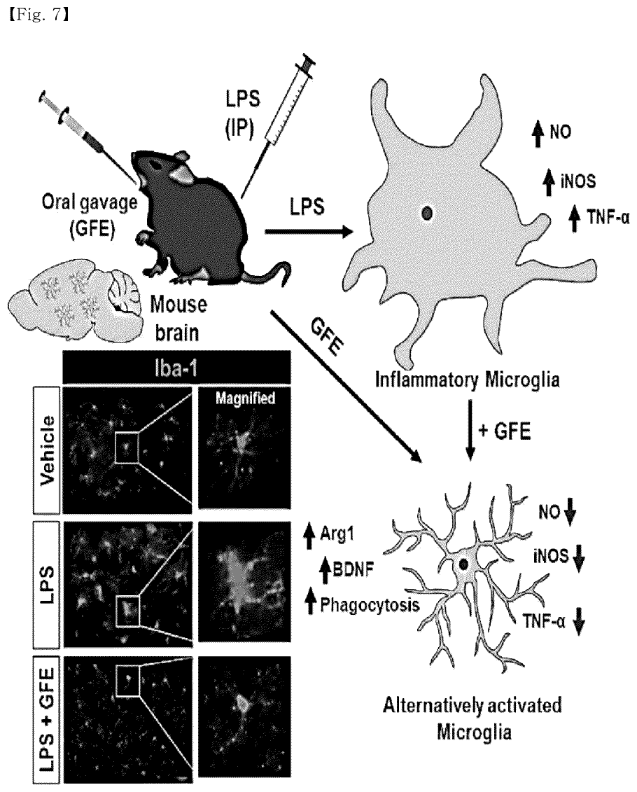

As a result, it was shown that the reduction in NO production by GFE (control 58.5%, PD 18%, and Bay 30.3%) was inhibited in MGCs pretreated with PD98059 and Bay 11-7082, respectively, so that the anti-inflammatory effect of GFE was inhibited (FIG. 6).

Through this, it was confirmed that the GFE inhibited the production of LPS-induced immune mediators through inactivation of the MAPK and NF-κ pathways.

1. A method for preventing or treating a neuroinflammatory disease, comprising administering to a subject in need thereof a pharmaceutical composition comprising a Daphne genkwa flower bud extract as an active ingredient.

2. The method of claim 1, wherein the extract is extracted with at least one solvent selected from the group consisting of water, an organic solvent, a subcritical fluid, and a supercritical fluid.

3. The method of neuroinflammatory disease of claim 1, wherein the extract is a methanol extract.

4. The method of neuroinflammatory disease of claim 1, wherein the neuroinflammatory disease is neuroinflammatory disease with increased activity of microglia or astrocytes.

5. The method of claim 1, wherein the subject requires inhibiting neuroinflammation in a cerebral cortex induced by activity of microglia or astrocytes.

6. A method for prevention or treatment of neurodegenerative disease, comprising administering to a subject in need thereof a Daphne genkwa flower bud extract as an active ingredient.

7. The method of claim 6, wherein the neurodegenerative disease is any one selected from the group consisting of Alzheimer's disease, Parkinson's disease, Creutzfeldt-Jakob disease (CJD), Hallervorden-Spatz disease, Huntington's disease, multiple system atrophy, dementia, frontotemporal dementia, amyotrophic lateral sclerosis, spinal muscular atrophy, spinocerebellar atrophy (SCA), meningoencephalitis, bacterial meningoencephalitis, viral meningoencephalitis, CNS autoimmune disorder, multiple sclerosis (MS), and acute ischemic injury.

8. The method of claim 6, wherein the neurodegenerative disease is neurodegenerative disease with increased activity of astrocytes or microglia.

9. A method for reducing activity of microglia in a neurodegenerative disease, comprising administering to a subject in need thereof a Daphne genkwa flower bud extract as an active ingredient.

10. The method of claim 9, wherein the subject requires inhibiting hyperactivation of the microglia in a cerebral cortex.

11. The method of claim 9, wherein the subject requires a protective effect against damage caused by activated microglia to nerve cells.

12. The method of claim 9, wherein the subject requires inhibiting expression of proinflammatory cytokines and inducible nitric oxide synthase (iNOS).

13. The method of claim 9, wherein the subject requires inhibiting production of nitric oxide (NO).

14. The method of claim 9, wherein the subject requires reducing expression and release of IL-1β in a cerebral cortex.

15. A method for improving or preventing a neuroinflammatory disease, comprising administering to a subject in need thereof a food composition comprising the Daphne genkwa flower bud extract of claim 9 as an active ingredient.

16. (canceled)
